(19) United States
(12) Patent Application Publication
(10) Pub. No.: US 2023/0029336 A1
(43) Pub. Date: Jan. 26, 2023

(54) Combination Therapy for Treating a Hematological Malignancy

(71) Applicant: AstraZeneca AB, Sodertalje (SE)

(72) Inventors: Maureen Hattersley, Wilmington, DE (US); Courtney Andersen, Wilmington (US); Huawei Raymond Chen, Wilmington (US)

(73) Assignee: AstraZeneca AB, Sodertalje (SE)

(21) Appl. No.: 17/768,130

(22) PCT Filed: Oct. 12, 2020
(86) PCT No.: PCT/IB2020/059556

§ 371 Date: Apr. 11, 2022

Related U.S. Application Data
(60) Provisional application No. 62/914,592, filed on Oct. 14, 2019.

(51) Int. Cl. A61K 31/5025; A61K 31/706; A61K 31/635; A61P 35/02

(52) U.S. Cl. CPC A61K 31/5025; A61K 31/706; A61K 31/635; A61P 35/02

(57) ABSTRACT

Disclosed are methods of treating cancer comprising administering to a subject in need thereof an effective amount of (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof and an effective amount of venetoclax or a pharmaceutically acceptable salt thereof.

### RELATED APPLICATION

This application claims benefit of priority under 35 U.S.C. § 119(e) of the U.S. Provisional Application No. 62/914,592, filed Oct. 14, 2019, which is incorporated by reference herein in its entirety for all purposes.

### BACKGROUND

While much progress has been made in the treatment of hematological malignancies, the many of these patients who have such cancers live with an incurable disease. Those patients suffering from acute myeloid leukemia (AML) have limited treatment options, and the five-year survival rate is approximately 25% with patients over 60 responding poorly to treatment, with a median survival of less than 12 months. Accordingly, it's important to continue to find new treatments for patients with incurable cancer.

### SUMMARY

In some embodiments, disclosed is a method of treating cancer, such as a hematological malignancy, comprising administering to a subject in need thereof an effective amount of AZD5153 or a pharmaceutically acceptable salt thereof and an effective amount of venetoclax or a pharmaceutically acceptable salt thereof. In some embodiments, the method further comprises administering an effective amount of 5-azacitidine.

In some embodiments, disclosed is AZD5153 or a pharmaceutically acceptable salt thereof for use in the treatment of cancer, such as a hematological malignancy, wherein said treatment comprises the separate, sequential or simultaneous administration of venetoclax or a pharmaceutically acceptable salt thereof. In some embodiments, the treatment further comprises administration of 5-azacitidine.

In some embodiments, disclosed is venetoclax or a pharmaceutically acceptable salt thereof for use in the treatment of cancer, such as a hematological malignancy, wherein said treatment comprises the separate, sequential or simultaneous administration of AZD5153 or a pharmaceutically acceptable salt thereof. In some embodiments, the treatment further comprises administration of 5-azacitidine.

In some embodiments, disclosed is a kit comprising: a first pharmaceutical composition comprising AZD5153 or a pharmaceutically acceptable salt thereof and a pharmaceutically acceptable carrier; a second pharmaceutical composition comprising venetoclax or a pharmaceutically acceptable salt thereof and a pharmaceutically acceptable carrier; and instructions for use. In some embodiments, the kit further comprises 5-azacitidine.

### BRIEF DESCRIPTIONS OF THE DRAWINGS

FIG. 1 illustrates the number of viable AML cells after 72 hours of treatment with vehicle, the combination of venetoclax (VEN) and 5-azacitidine (5-AZA), and the combination of venetoclax (VEN) and AZD5153 in a DFAM-68555 PDX model of AML.

FIG. 2 illustrates the reduction in tumor burden after 14 days of treatment with vehicle, the combination of venetoclax (VEN) and 5-azacitidine (5-AZA), and the combination of venetoclax (VEN) and AZD5153 in a DFAM-68555 PDX model of AML.

### DETAILED DESCRIPTION

In some embodiments, disclosed is a method of treating cancer by a combination therapy of AZD5153 and venetoclax and optionally 5-azacitidine. In some embodiments, the method comprises administering to a subject in need thereof an effective amount of AZD5153 or a pharmaceutically acceptable salt thereof and an effective amount of venetoclax or a pharmaceutically acceptable salt thereof. In some embodiments, the method further comprises administering 5-azacitidine.

The term “AZD5153” refers to a compound with the chemical name of (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one and structure shown below:

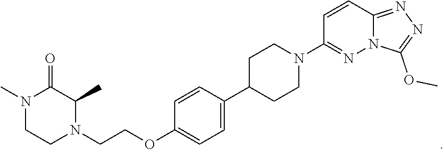

AZD5153 is a bivalent triazolopyridazine based bromodomain and extraterminal (BET) inhibitor. Bromodomain-containing proteins are implicated in diverse diseases and are of substantial interest as therapeutic targets. The BET family consists of four proteins known as BRD2, BRD3, BRD4, and BRDT, each of which contains two separate bromodomains. BRD4 in particular has been considered as a potential drug target for treating cancer (e.g., hematological malignancies).

Preparation and use of AZD5153 is disclosed in International Application Publication No. WO 2016/016618, the content of which is hereby incorporated by reference in its entirety. In some embodiments, a free base AZD5153, i.e., (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one, is administered to a subject. In some embodiments, a pharmaceutically acceptable salt of (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one is administered to a subject. In some embodiment, a crystalline AZD5153 is administered to a subject. In some embodiments, a co-crystal of AZD5153 is administered to a subject, wherein the co-former molecule is 6-hydroxy-2-naphthoic acid. Examples of crystalline AZD5153 and co-crystal of AZD5153 are described in WO 2016/016618.

In some embodiments, AZD5153 is administered orally. In some embodiments, AZD5153 is administered in a dose of up to about 40 mg (for example, up to about 5 mg, up to about 10 mg, up to about 15 mg, up to about 20 mg, up to about 25 mg, up to about 30 mg, up to about 35 mg, or up to about 40 mg AZD5153) either once a day (QD) or twice a day (BID). In some embodiments, AZD5153 is administered in a dose of 5 mg BID or 10 mg QD.

Venetoclax, also known as GDC-0199 or ABT-199, is described chemically as 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide) and has the following chemical structure:

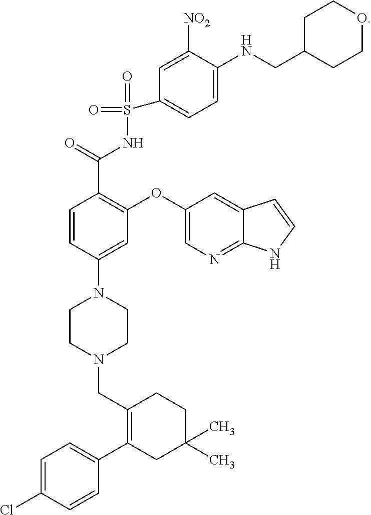 [SMILES: CC1(C)CCC(CN2CCN(c3ccc(C(=O)NS(=O)(=O)c4ccc(NCC5CCOCC5)c([N+](=O)[O-])c4)c(Oc4cnc5[nH]ccc5c4)c3)CC2)=C(c2ccc(Cl)cc2)C1]

Venetoclax is a selective and orally bioavailable inhibitor of BCL-2, an anti-apoptotic protein. Overexpression of BCL-2 has been demonstrated in CLL and AML cells where it mediates tumor cell survival and has been associated with resistance to chemotherapeutics. Venetoclax helps restore the process of apoptosis by binding directly to the BCL-2 protein, displacing pro-apoptotic proteins like BIM, triggering mitochondrial outer membrane permeabilization and the activation of caspases.

Preparation and use of venetoclax is disclosed in International Application Publication No. WO 2011/149492, the content of which is hereby incorporated by reference in its entirety. In some embodiments, a free base venetoclax is administered to a subject. In some embodiments, a pharmaceutically acceptable salt of venetoclax is administered to a subject. In some embodiment, a crystalline venetoclax or a pharmaceutically acceptable salt of venetoclax is administered to a subject.

In some embodiments, venetoclax or a pharmaceutically acceptable salt thereof is administered orally. In some embodiments, venetoclax or a pharmaceutically acceptable salt thereof is administered in a dose of up to about 600 mg (for example, up to about 20 mg, up to about 50 mg, up to about 100 mg, up to about 200 mg, up to about 400 mg, up to about 500 mg, or up to about 600 mg venetoclax) once a day (QD). In some embodiments, venetoclax or a pharmaceutically acceptable salt thereof is administered according to a weekly ramp-up schedule over 5 weeks to the daily dose of 400 mg. For example, or a pharmaceutically acceptable salt thereof is administered in a dose of 20 mg per day for Week 1, 50 mg per day for Week 2, 100 mg per day for Week 3, 200 mg per day for Week 4, and 400 mg per day for Week 5 and beyond.

The term “5-azacitidine” includes the compound of the structure:

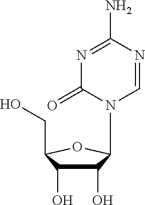

is also known as 4-amino-1-(β-D-ribofuranosyl)-1,3,5-triazin-2(1H)-one or ladakamycin, 5-azacytidine, azacitidine or azacytidine. 5-Azacitadine is thought to have antineoplastic activity via two mechanisms—at low doses, by inhibiting of DNA methyltransferase, causing hypomethylation of DNA, and at high doses, by its direct cytotoxicity to abnormal hematopoietic cells in the bone marrow through its incorporation into DNA and RNA, resulting in cell death. In some embodiments, the method comprises administering to the subject a pharmaceutical composition comprising 5-azacitidine and mannitol. In some embodiments, the pharmaceutical composition comprises a 1:1 weight ratio of 5-azacitidine and mannitol (e.g., 100 mg each of 5-azacitidine and mannitol). In some embodiments, 5-azacitidine is administered subcutaneously. In some embodiments, 5-azacitidine is administered intravenously. In some embodiments, the 5-azacitidine is administered at 75 mg/m² daily for 7 days, followed by repeat cycles every four weeks, with an increase of 100 mg/m². In some embodiments, 5-azacytadine is administered at a 75 mg/m² dose on day 1 through day 7 of a 28-day cycle. In some embodiments, 5-azacytinde is administered at a 75 mg/m² dose on day 1 through day 5 and days 8 and 9 of a 28-day (4-week) cycle.

In some embodiments, AZD5153 or a pharmaceutically acceptable salt thereof and venetoclax or a pharmaceutically acceptable salt thereof are administered in a coordinated fashion. In some embodiments, AZD5153 or a pharmaceutically acceptable salt thereof and venetoclax or a pharmaceutically acceptable salt thereof are administered separately, sequentially or simultaneously. In some embodiments, a subject in a treatment cycle is administered with AZD5153 or a pharmaceutically acceptable salt thereof without venetoclax or a pharmaceutically acceptable salt thereof for one or more days, and then is administered with both AZD5153 or a pharmaceutically acceptable salt thereof and venetoclax or a pharmaceutically acceptable salt thereof for the remainder days of the treatment cycle. Whilst not being bound by any theory, it is believed that AZD5153 or a pharmaceutically acceptable salt thereof, when administered to a subject first without venetoclax or a pharmaceutically acceptable salt thereof, can prime the subject for the treatment of administering both AZD5153 or a pharmaceutically acceptable salt thereof and venetoclax or a pharmaceutically acceptable salt thereof. In some embodiments, AZD5153 or a pharmaceutically acceptable salt thereof is administered for 1 to 14 days in a treatment cycle, and then both AZD5153 or a pharmaceutically acceptable salt thereof and venetoclax or a pharmaceutically acceptable salt thereof are administered for the remainder days of the treatment cycle. In some embodiments, AZD5153 or a pharmaceutically acceptable salt thereof is administered for 5 to 10 days in a treatment cycle, and then both AZD5153 or a pharmaceutically acceptable salt thereof and venetoclax or a pharmaceutically acceptable salt thereof are administered for the remainder days of the treatment cycle. In some embodiments, AZD5153 or a pharmaceutically acceptable salt thereof is administered for 5, 6, or 7 days in a treatment cycle, and then both AZD5153 or a pharmaceutically acceptable salt thereof and venetoclax or a pharmaceutically acceptable salt thereof are administered for the remainder days of the treatment cycle. In any of the preceding embodiments, the treatment cycle is on a weekly-basis and has 3 or more weeks in each treatment cycle. For example, the treatment cycle can be a 3-, 4-, 5-, 6-, 7-, 8-, 9-, 10-, 11-, 12-, or up to 52-week treatment cycle.

The language “treat,” “treating” and “treatment” includes the reduction or inhibition of enzyme or protein activity related to BRD4, DNA methyltransferase or cancer in a subject, amelioration of one or more symptoms of cancer in a subject, or the slowing or delaying of progression of cancer in a subject. The language “treat,” “treating” and “treatment” also includes the reduction or inhibition of the growth of a tumor or proliferation of cancerous cells in a subject.

The language “inhibit,” “inhibition” or “inhibiting” includes a decrease in the baseline activity of a biological activity or process.

The term “cancer” includes, but is not limited to hematological malignancies such as acute myeloid leukemia (AML), myelodysplastic syndrome (MDS), chronic myeloid leukemia (CML), chronic lymphocytic leukaemia (CLL), and chronic myelomonocytic leukemia (CMML). In some embodiments, the cancer includes cancers that are susceptible to treatment with BRD4 inhibitors (e.g., AZD5153). In some embodiments, the cancer includes cancers that are susceptible to treatment with BCL-2 inhibitors (e.g., venetoclax). In some embodiments, the hematological malignancies are relapsed or refractory hematological malignancies. In some embodiments, the hematological malignancies are resistant to a monotherapy of a BCL-2 inhibitor.

The language “pharmaceutical composition” includes compositions comprising an active ingredient and a pharmaceutically acceptable excipient, carrier or diluent, wherein the active ingredient is AZD5153 or a pharmaceutically acceptable salt thereof, or venetoclax or a pharmaceutically acceptable salt thereof or 5-azacitidine. The language “pharmaceutically acceptable excipient, carrier or diluent” includes compounds, materials, compositions, and/or dosage forms which are, within the scope of sound medical judgment, suitable for use in contact with the tissues of human beings and animals without excessive toxicity, irritation, allergic response, or other problem or complication, as ascertained by one of skill in the art. In some embodiments, the pharmaceutical compositions are in solid dosage forms, such as capsules, tablets, granules, powders, sachets, etc. In some embodiments, the pharmaceutical compositions are in the form of a sterile injectable solution in one or more aqueous or non-aqueous non-toxic parenterally-acceptable buffer systems, diluents, solubilizing agents, co-solvents, or carriers. A sterile injectable preparation may also be a sterile injectable aqueous or oily suspension or suspension in a non-aqueous diluent, carrier or co-solvent, which may be formulated according to known procedures using one or more of the appropriate dispersing or wetting agents and suspending agents. The pharmaceutical compositions could be a solution for iv bolus/infusion injection or a lyophilized system (either alone or with excipients) for reconstitution with a buffer system with or without other excipients. The lyophilized freeze-dried material may be prepared from non-aqueous solvents or aqueous solvents. The dosage form could also be a concentrate for further dilution for subsequent infusion.

The term “subject” includes warm-blooded mammals, for example, primates, dogs, cats, rabbits, rats, and mice. In some embodiments, the subject is a primate, for example, a human. In some embodiments, the subject is suffering from cancer, such as a hematological malignancy. In some embodiments, the subject is suffering from relapsed or refractory acute myeloid leukemia (AML). In some embodiments, the subject is suffering from relapsed or refractory high-risk myelodysplastic syndromes (MDS). In some embodiments, the subject is suffering from relapsed or refractory chronic myeloid leukemia (CML). In some embodiments, the subject is suffering from relapsed or refractory chronic lymphocytic leukaemia (CLL). In some embodiments, the subject is suffering from relapsed or refractory chronic myelomonocytic leukemia (CMML). In some embodiments, the subject is suffering from cancer and is treatment naïve (e.g., has never received treatment for cancer). In some embodiments, the subject is in need of treatment (e.g., the subject would benefit biologically or medically from treatment). In some embodiments, the subject is pretreated with anti-nausea medication.

The language “effective amount” includes that amount of AZD5153 and/or that amount of venetoclax and/or that amount of 5-azacitidine that will elicit a biological or medical response in a subject, for example, the reduction or inhibition of enzyme or protein activity related to BCL-2, BET including one or more of BRD2, BRD3, BRD4, and BRDT, or cancer; amelioration of symptoms of cancer; or the slowing or delaying of progression of cancer. In some embodiments, the language “effective amount” includes the amount of AZD5153 and/or venetoclax and/or 5-azacitidine that is effective to at least partially alleviate, inhibit, and/or ameliorate cancer or inhibit BRD4 or BCL-2, and/or reduce or inhibit the growth of a tumor or proliferation of cancerous cells in a subject.

In some embodiments, disclosed is a kit comprising: a first pharmaceutical composition comprising AZD5153 or a pharmaceutically acceptable salt thereof and a pharmaceutically acceptable carrier; and a second pharmaceutical composition comprising venetoclax or a pharmaceutically acceptable salt; and instructions for using the first and second pharmaceutical compositions in combination. In some embodiments, the kit further comprises a third pharmaceutical composition comprising 5-azacitidine.

In some embodiments, disclosed is a pharmaceutical product comprising i) (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof, and ii) venetoclax or a pharmaceutically acceptable salt thereof. In some embodiments of the pharmaceutical product, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof and venetoclax or a pharmaceutically acceptable salt thereof are present in a single dosage form. In some embodiments of the pharmaceutical product, (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof and venetoclax or a pharmaceutically acceptable salt thereof are present in separate dosage forms. In some embodiments, the pharmaceutical product further comprises 5-azacitidine, either in a single dosage form or separated dosage forms.

### EXAMPLE

### Example 1. Efficacy of AZD5153, a Bivalent BRD4 Inhibitor, Combined with Venetoclax in Preclinical Models of AML

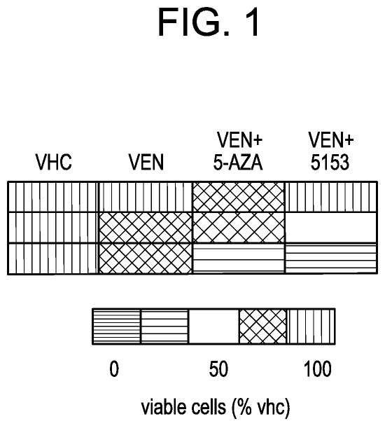

DFAM-68555 ex vivo screen: PDX cells were isolated from the spleen of female NSG mice bearing DFAM-68555 xenografts. PDX cells were resuspended in a 50/50 mixture of HS-5 cell conditioned media and IMDM+10% FBS. Cells were seeded in 96-well plates and treated with venetoclax, AZD5153+venetoclax, and 5-azacytidine+venetoclax. After 72 hrs of treatment, number of viable AML cells was assessed by flow cytometry. The result is shown in FIG. 1.

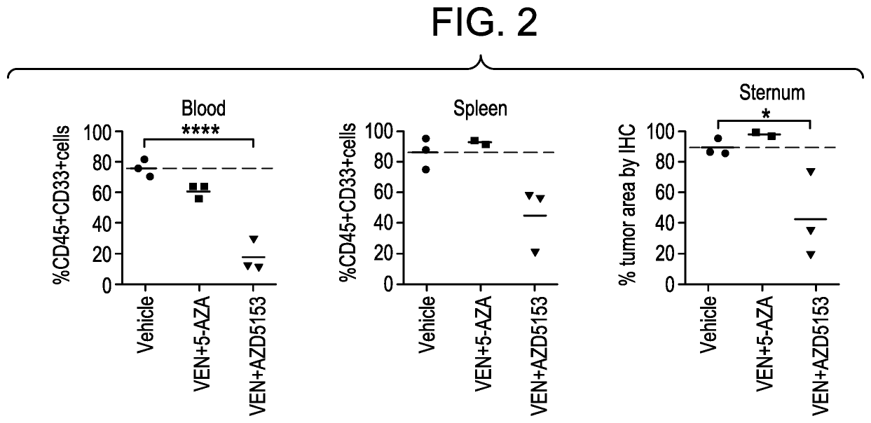

DFAM-68555 PDX efficacy: Female NSG mice were engrafted with 10⁶ DFAM-68555 PDX cells via tail vein injection. When circulating disease (measured by % huCD45+huCD33+ cells) reached ˜4%, mice were randomized into groups and treated with vehicle, venetoclax (100 mpk qd po)+5-azacytidine (1 mpk bid 3on/4off ip), or venetoclax (100 mpk qd po)+AZD5153 (0.075 mpk qd po). Venetoclax was formulated in 10% ethanol, 30% PEG-400, and 60% Phosal 50 PG. AZD5153 was formulated in 0.5% HPMC+0.1% Tween-80. Azacytidine was formulated in 0.9% saline. Animals were treated for 14 days. Circulating disease was monitored weekly by flow cytometry. At end of study, blood, spleen, and bone marrow (sternum) were collected for flow cytometry and histopathology analysis. The result is shown in FIG. 2.

Results: As shown in FIGS. 1 and 2, venetoclax+5-azacytidine had little to no efficacy in the DFAM-68555 PDX model ex vivo (FIG. 1) and in vivo (FIG. 2). However, venetoclax+AZD5153 significantly reduced tumor burden ex vivo and in vivo in the blood, spleen, and bone.

1. A method of treating a hematological malignancy comprising administering to a subject in need thereof an effective amount of (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof and an effective amount of venetoclax or a pharmaceutically acceptable salt thereof.

2. The method of claim 1, wherein the method comprises administering (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof sequentially, separately or simultaneously with venetoclax or a pharmaceutically acceptable salt thereof.

3. The method of claim 1 or 2, wherein the hematological malignancy is selected from acute myeloid leukemia (AML), myelodysplastic syndromes (MDS), chronic myeloid leukemia (CML), chronic lymphocytic leukaemia (CLL), and chronic myelomonocytic leukemia (CMML).

4. (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof for use in the treatment of a hematological malignancy, wherein the treatment comprises separate, sequential or simultaneous administration of venetoclax or a pharmaceutically acceptable salt thereof.

5. Venetoclax or a pharmaceutically acceptable salt thereof for use in the treatment of a hematological malignancy, wherein the treatment comprises separate, sequential or simultaneous administration of (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof.

6. The use of claim 4 or 5, wherein the hematological malignancy is selected from acute myeloid leukemia (AML), myelodysplastic syndromes (MDS), chronic myeloid leukemia (CML), chronic lymphocytic leukaemia (CLL), and chronic myelomonocytic leukemia (CMML).

7. The method or use of any one of claims 1 to 6, which further comprises administering an effective amount of 5-azacitidine.

8. The method or use of claim 3 or 6, wherein the hematological malignancy is a relapsed or refractory hematological malignancy.

9. The method or use of claim 3 or 6, wherein the hematological malignancy is resistant to a monotherapy of a BCL-2 inhibitor.

10. A kit comprising:
a first pharmaceutical composition comprising (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof and a pharmaceutically acceptable carrier; and
a second pharmaceutical composition comprising venetoclax or a pharmaceutically acceptable salt thereof and instructions for use.

11. The kit of claim 10, which further comprises a third pharmaceutical composition comprising 5-azacitidine.

12. A pharmaceutical product comprising i) (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof, and ii) venetoclax or a pharmaceutically acceptable salt thereof.

13. The pharmaceutical product of claim 1, wherein (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof and venetoclax or a pharmaceutically acceptable salt thereof are present in a single dosage form.

14. The pharmaceutical product of claim 1, wherein (3R)-4-[2-[4-[1-(3-Methoxy-[1,2,4]triazolo[4,3-b]pyridazin-6-yl)-4-piperidyl]phenoxy]ethyl]-1,3-dimethyl-piperazin-2-one or a pharmaceutically acceptable salt thereof and venetoclax or a pharmaceutically acceptable salt thereof are present in separate dosage forms.
